(19) United States
(12) Patent Application Publication
(10) Pub. No.: US 2022/0126111 A1
(43) Pub. Date: Apr. 28, 2022

(54) HANDHELD MILLIMETER WAVE IRRADIATION DEVICE

(71) Applicants: Keng-Yuan CHEN, Keelung City (TW); Man-Chee LIU, Taipei City (TW)

(72) Inventor: Keng-Yuan CHEN, Keelung City (TW)

(21) Appl. No.: 17/505,998

(22) Filed: Oct. 20, 2021

(30) Foreign Application Priority Data
Oct. 26, 2020 (TW) 109214090

(51) Int. Cl. A61N 5/06

(52) U.S. Cl. CPC A61N 5/06; A61N 2005/0658; A61N 2005/0644

(57) ABSTRACT

A handheld millimeter wave irradiation device including: a housing having a holding part, an irradiation part, and an operation control part; and at least one circuit board and a battery unit disposed inside the housing, the battery unit being used for supplying power to the at least one circuit board; where the holding part is used to facilitate hand holding for a user, the irradiation part is used to aim at a selected area of a human body for a millimeter wave irradiation operation, and the operation control part is used for the user to input commands to turn on/off the handheld millimeter wave irradiation device and select an operation mode of the millimeter wave irradiation operation.

### BACKGROUND OF THE INVENTION

### Field of the Invention

The invention is related to a human body treatment irradiation device, especially to a handheld millimeter wave irradiation device.

### Description of the Related Art

In the field of physical therapy today, common equipment includes ultrasonic device, far-infrared radiation device, intermediate frequency interference wave therapy device, transcutaneous neuromuscular electrical stimulator, and so on.

Although those treatment devices have certain effects, however, the treatment process thereof also brings some discomfort, such as burning sensation and muscle cramps. In addition, those treatment devices usually have a large size, which is not easy to carry.

To solve the aforementioned problems, a novel human body irradiation device is needed in the art.

### SUMMARY OF THE INVENTION

One objective of the invention is to disclose a handheld millimeter wave irradiation device, which can enable a user to irradiate any part of the user's body.

Another objective of the invention is to disclose a handheld millimeter wave irradiation device, which can enable a user to select an appropriate operation mode to meet the user's need.

Still another objective of the invention is to disclose a handheld millimeter wave irradiation device, which can provide a radiation treatment without causing a burning sensation on the body.

To achieve the aforementioned objectives, a handheld millimeter wave irradiation device is proposed, which includes:

a housing having a holding part, an irradiation part and an operation control part; and

at least one circuit board and a battery unit located inside the housing, the battery unit being used for supplying power to the at least one circuit board;

where, the holding part is used to facilitate hand holding for a user, the irradiation part is used to aim at a selected area of a human body for a millimeter wave irradiation operation, and the operation control part is used for the user to input commands to control the power on/ff of the handheld millimeter wave irradiation device and select an operation mode of the millimeter wave irradiation operation.

For possible embodiments, the operation mode may determine a power size of the irradiation, an irradiation time or an irradiation manner.

For possible embodiments, the irradiation manner can be continuous or intermittent irradiation.

In a possible embodiment, the housing may further have a connection port for connecting with an external power source to charge the battery unit.

In one embodiment, the connection port is a USB connection interface.

For possible embodiments, the operation control part may include a keypad device or a touch device.

In one embodiment, the at least one circuit board includes:

a millimeter-wave radiation unit, a high frequency signal generating unit, and at least one antenna unit, the millimeter-wave radiation unit is coupled between the high frequency signal generating unit and the at least one antenna unit, and is configured to drive the at least one antenna unit to generate a millimeter wave radiation according to a high frequency signal generated by the high frequency signal generating unit; and

a central processing unit coupled with the high frequency signal generating unit and the operation control part to drive the high frequency signal generating unit according to a command from the operation control part to determine the operation mode.

In an embodiment, the at least one circuit board further includes:

a communication unit coupled with the central processing unit for communicating with an external device wirelessly, whereby the central processing unit can receive operation control of the external device or transmit an operation record to the external device.

For possible embodiments, the communication unit can be a WiFi communication unit or a Bluetooth communication unit.

In one embodiment, the battery unit has a battery and a power supply unit, and the power supply unit is coupled between the connection port and the battery for generating a DC voltage to power the at least one circuit board, and for charging the battery by using power from an external power supply connected with the connection port.

To make it easier for our examiner to understand the objective of the invention, its structure, innovative features, and performance, we use preferred embodiments together with the accompanying drawings for the detailed description of the invention.

### BRIEF DESCRIPTION OF THE DRAWINGS

FIG. 1 illustrates a schematic diagram of a handheld millimeter wave irradiation device according to one embodiment of the invention;

FIG. 2 illustrates an exploded diagram of the handheld millimeter wave irradiation device of FIG. 1; and

FIG. 3 illustrates a functional block diagram of the handheld millimeter wave irradiation device of FIG. 1.

### DETAILED DESCRIPTION OF THE PREFERRED EMBODIMENTS

The principle of the invention lies in:

Using millimeter waves to irradiate the human body to make the stimulated cells resonant can produce a non-thermal biological effect to provide a treatment similar to acupuncture. As the treatment does not rely on heat, there will be no scalding problem.

Millimeter waves refer to electromagnetic waves with a wavelength of 1 mm to 10 mm, which can interact with organisms to produce special biological effects. Specifically, the oscillation frequency generated in the millimeter wave band is similar to the natural frequency or characteristic frequency of human macromolecular cells. The resonance generated by the interaction between the two will significantly increase the activity and quality of the cells, thereby exciting the vitality of the immune system of human body to effectively resist and inhibit the growth of viruses in the body.

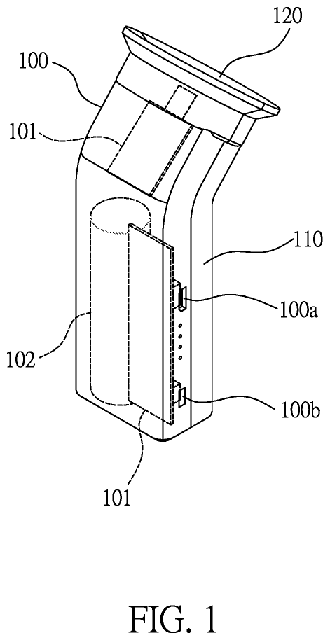

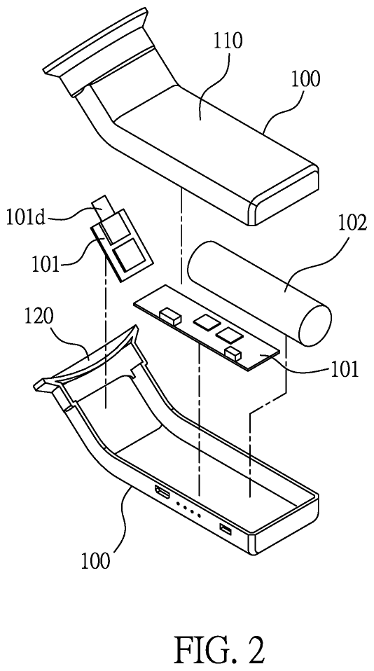

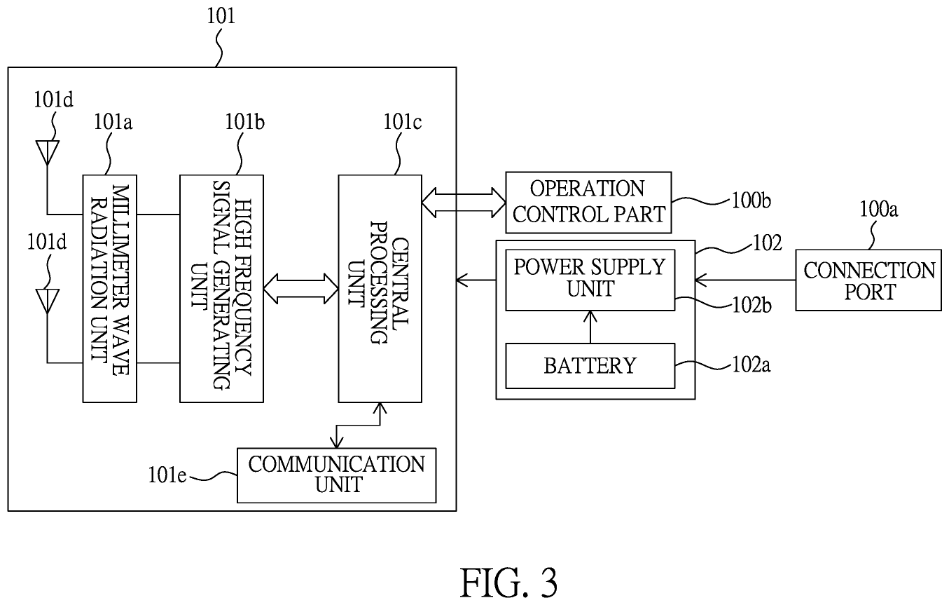

Please refer to FIGS. 1-3, where FIG. 1 illustrates a schematic diagram of a handheld millimeter wave irradiation device according to one embodiment of the invention; FIG. 2 illustrates an exploded diagram of the handheld millimeter wave irradiation device of FIG. 1; and FIG. 3 illustrates a functional block diagram of the handheld millimeter wave irradiation device of FIG. 1.

As shown in FIGS. 1-2, the handheld millimeter wave irradiation device of the present invention has a housing 100 with a holding part 110, an irradiation part 120, a connection port 100a and an operation control part 100b. The housing 100 has at least one circuit board 101 and a battery unit 102 for supplying power to the at least one circuit board 101.

The holding part 110 is used to facilitate holding for a user.

The irradiation part 120 is used to aim at a selected area of a human body to perform a millimeter wave irradiation operation.

The connection port 100a is used to connect to an external power source to charge the battery unit 102. In a possible embodiment, the connection port 100a can be a USB connection interface to connect to a USB power source.

The operation control part 100b may include a keypad device or a touch device for a user to input commands to turn on/off of the handheld millimeter wave irradiation device and select an operation mode of the millimeter wave irradiation operation, where the operation mode may determine a power size of the irradiation, an irradiation time or an irradiation manner (for example, continuous or intermittent irradiation).

In addition, as shown in FIG. 3, the at least one circuit board 101 has a millimeter wave radiation unit 101a, a high frequency signal generating unit 101b, a central processing unit 101c, at least one antenna unit 101d, and a communication unit 101e, and is powered by the battery unit 102. The battery unit 102 has a battery 102a and a power supply unit 102b. The power supply unit 102b is coupled between the connection port 100a and the battery 102a for generating a DC voltage to power the at least one circuit board 101, and for charging the battery 102a by using power from an external power supply connected with the connection port 100a.

The millimeter wave radiation unit 101a is coupled between the high frequency signal generating unit 101b and the at least one antenna unit 101d, and is configured to drive the at least one antenna unit 101d to generate a millimeter wave radiation according to a high frequency signal generated by the high frequency signal generating unit 101b.

The central processing unit 101c is coupled with the high frequency signal generating unit 101b and the operation control part 100b to determine the operating mode according to the commands of the operation control part 100b, thereby determining the power size of the irradiation, the irradiation time or the irradiation manner (for example, continuous or intermittent irradiation) according to the user's input.

In addition, the central processing unit 101c can also wirelessly communicate with an external device through the communication unit 101e to receive operation commands from the external device or transmit an operation record to the external device, where the communication unit 101e can be a WiFi communication unit or a Bluetooth communication unit, and the external device can directly communicate with the communication unit 101e wirelessly or communicate with the communication unit 101e via a network.

Thanks to the designs disclosed above, the invention possesses the following advantages:

1. The handheld millimeter wave irradiation device of the invention can enable a user to irradiate any part of the user's body.

2. The handheld millimeter wave irradiation device of the invention can enable a user to select an appropriate operation mode to meet the user's need.

3. The handheld millimeter wave irradiation device of the invention can provide a radiation treatment without causing a burning sensation on the body.

While the invention has been described by way of example and in terms of preferred embodiments, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

In summation of the above description, the present invention herein enhances the performance over the conventional structure and further complies with the patent application requirements and is submitted to the Patent and Trademark Office for review and granting of the commensurate patent rights.

What is claimed is:

1. A handheld millimeter wave irradiation device, comprising:
a housing having a holding part, an irradiation part and an operation control part; and
at least one circuit board and a battery unit located inside the housing, the battery unit being used for supplying power to the at least one circuit board;
wherein, the holding part is used to facilitate hand holding for a user, the irradiation part is used to aim at a selected area of a human body for a millimeter wave irradiation operation, and the operation control part is used for the user to input commands to control the power on/ff of the handheld millimeter wave irradiation device and select an operation mode of the millimeter wave irradiation operation.

2. The handheld millimeter wave irradiation device as disclosed in claim 1 of the scope of the patent application, wherein the operation mode is used to determine a power size of the irradiation, an irradiation time or an irradiation manner.

3. The handheld millimeter wave irradiation device as disclosed in claim 2, wherein the irradiation manner can be continuous or intermittent irradiation.

4. The handheld millimeter wave irradiation device as disclosed in claim 1, wherein the housing further includes a connection port for connecting with an external power source to charge the battery unit.

5. The handheld millimeter wave irradiation device as disclosed in claim 4, wherein the connection port is a USB connection interface.

6. The handheld millimeter wave irradiation device as disclosed in claim 1, wherein the operation control part includes a keypad device or a touch device.

7. The handheld millimeter wave irradiation device as disclosed in claim 1, wherein the at least one circuit board includes:
a millimeter-wave radiation unit, a high frequency signal generating unit, and at least one antenna unit, the millimeter-wave radiation unit is coupled between the high frequency signal generating unit and the at least one antenna unit, and is configured to drive the at least one antenna unit to generate a millimeter wave radiation according to a high frequency signal generated by the high frequency signal generating unit; and
a central processing unit coupled with the high frequency signal generating unit and the operation control part to drive the high frequency signal generating unit according to a command from the operation control part to determine the operation mode.

8. The handheld millimeter wave irradiation device as disclosed in claim 7, wherein the at least one circuit board further includes:
a communication unit coupled with the central processing unit for communicating with an external device wirelessly, whereby the central processing unit can receive operation control of the external device or transmit an operation record to the external device.

9. The handheld millimeter wave irradiation device as disclosed in claim 8, wherein the communication unit is a WiFi communication unit or a Bluetooth communication unit.

10. The handheld millimeter wave irradiation device as disclosed in claim 4, wherein the battery unit has a battery and a power supply unit, and the power supply unit is coupled between the connection port and the battery for generating a DC voltage to power the at least one circuit board, and for charging the battery by using power from an external power supply connected with the connection port.
